SEQUENCE LISTING (9 sequences)

SEQ ID NO: 1 - DNA, 20 bp, Homo sapiens
ccactccagacccaggaatg

SEQ ID NO: 2 - DNA, 20 bp, Homo sapiens
tgtctggccttctggagcat

SEQ ID NO: 3 - DNA, 23 bp, Homo sapiens
tcccatgccttcaccactcccaa

SEQ ID NO: 4 - DNA, 19 bp, Homo sapiens
ctacggcgctgtcatcgat

SEQ ID NO: 5 - DNA, 25 bp, Homo sapiens
tggagcttattaaaggcattcttca

SEQ ID NO: 6 - DNA, 27 bp, Homo sapiens
cttccctgtgaaaacaagagcaaggcc

SEQ ID NO: 7 - DNA, 21 bp, Homo sapiens
ccttcagtagcaccggaatca

SEQ ID NO: 8 - DNA, 25 bp, Homo sapiens
caaacaaacacacaggcatgtagtt

SEQ ID NO: 9 - DNA, 20 bp, Homo sapiens
agcctgcagccgctccagcc

(19) United States
(12) Patent Application Publication
(10) Pub. No.: US 2005/0159425 A1
(43) Pub. Date: Jul. 21, 2005

(54) Methods of therapy

(76) Inventor: Rodney William Kelly, Edinburgh (GB)

Correspondence Address: Edwin V Merkel;Nixon Peabody, Clinton Square, PO Box 31051, Rochester, NY, 14603, US

(21) Appl. No.: 10/513,998

(22) PCT Filed: May 9, 2003
(86) PCT No.: PCT/GB03/02009

(30) Foreign Application Priority Data
May 10, 2002 (GB) 0210741.5

(51) Int. Cl. A61K 31/522; A61K 31/519; A61K 31/4015; A61K 31/557

(52) U.S. Cl. 514/252.16; 514/263.31; 514/424; 514/573

(57) ABSTRACT

A method of inducing tolerance to an antigen in a patient, the method comprising administering to the patient a prostaglandin or agonist thereof, a phosphodiesterase inhibitor and the antigen or a derivative thereof.

The present invention relates to the rapeutic methods and uses in particular it relates to methods for inducing tolerance to an antigen in a patient.

An organism's immunity to an antigen arises as a consequence of a first encounter with the antigen and the subsequent production of immunoglobulin molecules, for example, antibodies, capable of selectively binding that antigen. In addition, the immune response is controlled by T cells which may be antigen specific. Immunity allows the rapid recruitment, usually by stimulating an inflammatory response, of cells which can dispose of the foreign antigen. Under certain circumstances, the immune system does not produce an immune response against antigens due to a mechanism called “tolerance”. For example, an immune system can normally discriminate against foreign antigens and constituents of the organism itself, due to a mechanism whereby all B lymphocytes which could potentially produce antibodies to constituents of the organism itself (“self antigens”) are destroyed during development, thereby removing the organism's capacity to produce antibodies directed to a self antigen.

Tolerance is probably an active process. This means that peripheral tolerance is gained where an antigen is presented to a T cell in a particular tolerising environment (eg high IL-10 levels and low IL-12 levels). The T cells then circulate and when they meet that specific antigen again they do not mount an immune response (anergic T cells) or they mount a quelling response (regulatory T cells). A role for regulatory T cells has been proposed in tolerance. The regulatory T cells are programmed by the environment of the antigen presenting cell to react to their cognate antigen by releasing “down-regulatory” cytokines. The first such regulatory cells described were induced by IL-10 (Groux et al., 1997, Nature 389: 737-742).

Where tolerance breaks down, the organism may produce a cellular immune response (including cytotoxic T cells) to normal constituents of the organism, producing an “autoimmune disease”. Examples of autoimmune diseases include systemic lupus erythematosus (SLE), multiple sclerosis (MS) and Hashimoto's disease.

In some circumstances, even the normal response of the immune system to a foreign antigen can produce undesirable results, such as in the case of tissue or organ grafts or transplants, where the immune system of the tissue or organ recipient recognises the tissue or organ graft or transplant as foreign and acts to reject it.

One of the drawbacks of existing methods of treating immune or inflammatory conditions or diseases however, is the limited range of options and their therapeutic inadequacy. For example, glucocorticosteroids used for treating inflammatory respiratory disease have toxic effects in many patients, and alternatives such as cyclosporin A or interferon γ are high-risk, expensive and generally unsatisfactory.

Unexpectedly, the inventor has found that there is a synergistic effect between prostaglandin and a phosphodiesterase (PDE) inhibitor on the release of interleukin-10 (IL-10) from cells of the immune system. Furthermore, the inventor has found that there is a marked stimulation of IL-10 and inhibition of interleukin-12 (IL-12) in cells of the immune system when a prostaglandin and a PDE inhibitor are used in combination. In the presence of a PDE inhibitor, the stimulation of IL-10 by both PGE and 19-hydroxy PGE was increased strikingly, resulting in a tolerising environment.

DE inhibitors such as Rolipram are known to raise cAMP and IL-10 levels in monocyte/macrophages stimulated with the bacterial coat product lipopolysaccharide (LPS) (Strassman et al., 1994 J. Exp. Med. 180: 2365-70; Kraan et al., 1995 J. Exp. Med. 181: 775-9; Kambayashi et al., 1995 J. Immunol. 155: 4909-16).

The inventor also shows an increase in PDE activity that follows both PGE and 19-hydroxy PGE application. This is a direct negative feedback to reduce the effect of the stimulus. Use of a PGE and a PDE inhibitor increases PDE message even further, but then the synthesised phosphodiesterase is nullified by the presence of the inhibitor.

In diseases resulting from an aberrant or undesired immune response there is often a deficiency in IL-10 and/or an increase in IL-12. This imbalance in IL-10 may be detrimental to the development of useful T helper cells, particularly T helper cells; a preponderance of type 1 T helper cells over type 2 T helper cells is thought to be characteristic of autoimmune disease. Thus, stimulation of IL-10 production and inhibition of IL-12 is believed to induce a tolerising environment for T cell activation. In addition, a high IL-10 and low IL-12 environment will act on an antigen presenting cell (typically a dendritic cell) to ensure regulatory T cell formation, creating a regulatory T cell that is specific for the antigen presented.

The inventor now proposes inducing tolerance to a desired antigen in a patient by the use of a PDE inhibitor in combination with a prostaglandin or agonist thereof to induce a tolerising environment in the patient, and administering the desired antigen or a derivative thereof to the patient.

Furthermore, the combination of a PDE inhibitor, prostaglandin or agonist thereof and antigen or derivative thereof is considered by the inventor to achieve the desirable effect of reducing the amount of prostaglandin or agonist thereof or PDE inhibitor required to achieve a useful degree of therapeutic benefit, and/or reducing the side effects of administration of prostaglandin or agonist thereof.

As far as the inventor is aware, there has never been any suggestion that a combination of a prostaglandin or agonist thereof and an inhibitor of PDE could be used to stimulate IL-10 production, and there has been no suggestion of a treatment using this combination to stimulate IL-10. Furthermore, there has never been any suggestion that this combination could be used to inhibit IL-12 production, or to induce a tolerising environment for T cell activation, or to induce tolerance to an antigen in a patient. Furthermore, there has never been any suggestion that administering an antigen or derivative thereof, prostaglandin and PDE inhibitor to the patient could be used to induce tolerance to that antigen in the patient.

The principal receptors for prostaglandin E2 (PGE2) are the EP2 and EP4 sub-types; however, other receptor sub-types exist (namely EP1 and EP3). EP2 and EP4 receptors couple with adenylcyclase and use elevated cAMP as the messenger system. The levels of cAMP in tissue are governed both by synthesis and by catabolism by PDE. PDE can be blocked by specific inhibitors. The inventor believes, but without being bound by any theory, that the administration of a PDE inhibitor will enhance the effect of a prostaglandin or agonist thereof in inducing tolerance to a specific desired antigen that is administered to a patient. Thus, the inventor believes, but without being bound by any theory, that the effect of a prostaglandin or agonist thereof (such as PGE) acting on its EP2 and EP4 receptors is to stimulate cAMP and the addition of the PDE inhibitor provides a synergistic action on monocytes and macrophages resulting in a reduction in the immune and/or inflammatory response which is greater than the effect of the sum of the same amount of either prostaglandin or agonist thereof or PDE inhibitor administered alone.

A first aspect of the invention provides a method of inducing tolerance to an antigen in a patient, the method comprising administering to the patient a prostaglandin or agonist thereof, a PDE inhibitor and the antigen, or derivative thereof.

The antigen or derivative thereof administered is typically the antigen to which it is desired to induce tolerance or a derivative thereof.

By inducing tolerance to an antigen we include the meaning that the immune system of the patient may become tolerant of an antigen where it was intolerant before, or the immune system may mount a reduced response or no response at all (ie, an undetectable response) to the antigen.

It is appreciated that to induce tolerance to an antigen, a derivative of the antigen may be administered to the patient, and not the antigen itself. By “derivative” of an antigen we include any portion of the antigen which can be presented by a class I or a class II MHC molecule for example on an antigen presenting cell (APC), and which induces tolerance to the antigen itself. Typically the derivative of the antigen is also recognised by a T cell when presented, for example via a T cell receptor.

When the antigen is a protein, a derivative of the antigen is typically a peptide fragment of the antigen consisting of a contiguous sequence of amino acids of the antigen capable of MHC binding. Preferably, the fragment is between 6 and 100 amino acids in length. More preferably, the fragment is between 6 and 50 amino acids in length. Most preferably, the fragment is six, or seven, or eight, or nine, or ten, or eleven, or twelve, or thirteen, or fourteen, or fifteen, or sixteen, or seventeen, or eighteen, or nineteen, or twenty, or twenty-one, or twenty-two, or twenty-three, or twenty-four or twenty-five amino acids in length.

A derivative of the antigen may include a fusion of the antigen, or a fusion of a fragment of the antigen, to another compound, and which can be recognised by either a class I or a class II MHC molecule when presented, and which induces tolerance to the antigen itself. Typically, the fusion is one which can be processed by an APC so as to present a portion which is able to induce tolerance to the antigen itself.

Unless the context indicates otherwise, wherever the term “antigen” is used in the context of an administered antigen, a derivative as herein defined is included.

The invention includes suppressing the immune system or an inflammatory response in a patient. By “suppressing” we include the meaning that the immune system or the inflammatory response is altered such that, in the case of an inflammatory response, a reduced inflammatory response to a stimulus is obtained, or an inflammatory response is avoided to the extent that a response is undetectable.

Accordingly, the invention includes inducing tolerance to an antigen in a patient to treat an aberrant or undesired immune or inflammatory response in the patient. This may be particularly useful in the treatment of diseases or conditions where there is an undesirable inflammatory response or immune response. The invention therefore includes a method of suppressing an immune response or an inflammatory response in a patient, the method comprising administering to the patient a prostaglandin or agonist thereof, a PDE inhibitor, and an antigen to which it is desired to induce tolerance or a derivative thereof.

By “aberrant or undesired immune or inflammatory response” we include diseases or conditions which cause the presence of visible or measurable inflammation within a tissue in an individual or patient. For example, the tissue that forms part of an allograft or the tissues of a host having received an allograft, or the central nervous system of an individual with MS, or insulitis in a patient with type 1 diabetes, or swollen joints in a patient with rheumatoid arthritis.

The invention includes a method of inducing tolerance to an antigen in a patient thereby suppressing an aberrant or undesired immune or inflammatory response in the patient, such as a response related to transplant rejection.

The invention includes a method of combating transplant rejection, or a disease or condition associated with transplant rejection, in a patient, the method comprising administering to the patient a prostaglandin or agonist thereof, a PDE inhibitor, and an antigen to which it is desired to induce tolerance, or a derivative thereof. The disease or condition associated with transplant rejection may be graft versus host disease, in which typically the antigen is a host antigen, ie an antigen present in the transplant recipient. Alternatively, the disease or condition associated with transplant rejection may be host versus graft disease in which typically the antigen is one which is present on the transplanted organ or material. In these cases, an inhibition or dampening of an immune or inflammatory response associated with an increase in T regulatory cells specific for antigens in the transplant may be required. Preferably the antigen is a class I MHC molecule. Most preferably, the MHC molecule is HLA-A2.

Diseases or conditions where there is an aberrant or undesired immune or inflammatory response may also include allergies, wherein the undesired response is an allergic response. In such a condition or disease, the antigen to which tolerance is induced would be an allergen.

Thus the methods of the invention may be particularly useful in the treatment of an allergic condition or disease where there is an undesirable allergic inflammatory or immune response.

The invention includes a method of treating, preventing or suppressing an allergic response in a patient, the method comprising administering to the patient a prostaglandin or agonist thereof, a PDE inhibitor, and an antigen to which it is desired to induce tolerance or a derivative thereof. Typically, in an allergic condition or disease, the antigen to which tolerance is induced would be an allergen.

In one preferred embodiment, the allergic condition or disease is allergic asthma. Preferably, the antigen and/or the PG and/or the PDE inhibitor are administered to the lungs or bronchial tree via an aerosol. Most preferably, the antigen and the prostaglandin and the PDE inhibitor are administered via an aerosol. This embodiment may be particularly advantageous as some 19-hydroxy prostaglandin analogues have been reported to function as bronchodilators, such as those described in U.S. Pat. No. 4,127,612, incorporated herein by reference. The reason why prostaglandins are not widely used in the treatment of asthma is that they make the patient cough. Administration of a PDE inhibitor would allow the prostaglandin to be administered at a lower concentration, thus providing the therapeutic benefits while minimising the side-effects.

Thus the invention includes the use of a 19-hydroxy PGE, a phosphodiesterase inhibitor, and an allergen to which it is desired to induce tolerance for treatment by inhalation of allergic asthma, or a derivative thereof.

In one preferred embodiment, if the disease or condition is an allergic disease or condition, such as allergic asthma, the antigen may be a mite allergen, a dust allergen, or a mammalian allergen such as a cat or a dog or a horse allergen, preferably a cat allergen.

In other embodiments, the antigen (allergen) may be any of the following: Fe1 d 1 (the feline skin and salivary gland allergen of the domestic cat Felis domesticus—the amino acid sequence of which is disclosed in WO 91/06571); Der p I, Der p II, Der fI or Der fII (the major protein allergens from the house dust mite dermatophagoides—amino acid sequences disclosed in WO 94/24281); and allergens present in any of the following: grass, tree and weed (including ragweed) pollens; fungi and moulds; foods eg fish, shellfish, crab lobster, peanuts, nuts, wheat gluten, eggs and milk; stinging insects eg bee, wasp and hornet and the chirnomidae (non-biting midges); spiders and mites, including the house dust mite; allergens found in the dander, urine, saliva, blood or other bodily fluid of mammals such as cat, dog, cows, pigs, sheep, horse, rabbit, rat, guinea pig, mouse and gerbil; airborne particulates in general; latex; and protein detergent additives.

The antigen (allergen) may also be an insect antigen, selected from the group of insects comprising: housefly, fruit fly, sheep blow fly, screw worm fly, grain weevil, silkworm, honeybee, non-biting midge larvae, bee moth larvae, mealworm, cockroach and larvae of Tenibrio molitor beetle.

In another embodiment, the invention includes a method of treating an autoimmune disease in a patient, the method comprising administering to the patient a prostaglandin or agonist thereof, a PDE inhibitor, and an antigen to which it is desired to induce tolerance or a derivative thereof. Typically, the antigen is a self-antigen against which there is an undesired immune response.

Preferably, if the disease or condition is pernicious anaemia, the antigen may be vitamin B₁₂.

Preferably, if the disease or condition is Addison's disease, the antigen may be adrenal antigen.

Preferably, if the disease or condition is insulin-dependent diabetes mellitus (IDDM), the antigen may be glutamic acid decarboxylase (GAD), insulin, or IA-2 (a protein tyrosine phosphatase-like molecule).

Preferably, if the disease or condition is Goodpasture's syndrome or renal vasculitis, the antigen may be renal antigen or endothelial antigen.

Preferably, if the disease or condition is myasthenia gravis, the antigen may be the acetyl choline receptor

Preferably, if the disease or condition is sympathetic ophthalmia, the antigen may be ocular antigen.

Preferably, if the disease or condition is MS, the antigen may be MBP (myelin basic protein), PLP (proteolipid protein), or MOG (myelin oligodendrocyte glycoprotein).

Preferably, if the disease or condition is autoimmune haemolytic anaemia, the antigen may be red cell antigen.

Preferably, if the disease or condition is idiopathic leucopenia, the antigen may be leukocyte antigen.

Preferably, if the disease or condition is ulcerative colitis, the antigen may be a food antigen or a viral antigen.

Preferably, if the disease or condition is dermatomyositis, the antigen may be smooth muscle antigen.

Preferably, if the disease or condition is scleroderma, the antigen may be connective tissue antigen.

Preferably, if the disease or condition is mixed connective tissue disease, the antigen may be connective tissue antigen.

Preferably, if the disease or condition is irritable bowel syndrome, the antigen may be a food antigen.

Preferably, if the disease or condition is SLE, the antigen may be histone proteins or immunoglobulin heavy chain.

Preferably, if the disease or condition is Hashimoto's disease, primary myxoedema or thyrotoxicosis the antigen may be thyroid antigen.

Preferably, if the disease or condition is thyroid autoimmune disease or thyroiditis, the antigen may be a thyroid hormone such as thyroglobulin.

Preferably, if the disease or condition is Behcet's disease, the antigen may be Sag (S antigen from the eye), HLA-B44, B51, or HSP65.

Preferably, if the disease or condition is Coeliac disease/Dermatitis herpetiformis, the antigen may be gliadin. Rather than use whole gliadin, it may be useful to use a fraction of gliadin which is able to down regulate gliadin-specific T-cell proliferation. A suitable fraction may be the a fraction disclosed in Maurano et al (2001) Scand. J. Immunol. 53, 290-295, incorporated herein by reference.

Preferably, if the disease or condition is rheumatoid arthritis, the antigen may be type II collagen or an HSP (heat shock protein).

Preferably, if the disease or condition is demyelinating disease, the antigen may be myelin.

The methods of the invention can be used to retolerise a patient to an antigen. For example, in an autoimmune disease or condition which is a result of a viral infection, the antigen may be a self-HSP that is similar to a viral HSP.

The treatment is believed to combat the undesirable autoimmune response directly, as well as treating the symptoms by directing T cells away from a pro-inflammatory role.

Without being bound by theory, the inventor believes that the methods of the present invention may affect the programming of T cells so that they become regulatory or suppressive T cells rather than pro-inflammatory T cells. When a T cell meets an antigen, in the presence of a prostaglandin and PDE inhibitor, it will release a suppressive cytokine such as IL-10 and not an inflammatory cytokine such as IL-12. Treatment with a prostaglandin and PDE inhibitor is thus believed to prevent or minimise an inflammatory response to that antigen from developing. Thus treatment with a prostaglandin, PDE inhibitor and an antigen, eg a self-antigen, or a derivative thereof, can be used prophylactically, or as soon as the first symptoms of, eg an autoimmune disease, appear.

Furthermore, it will be appreciated that because T cells are present throughout the body they may be programmed or primed at a site remote from their ultimate site of action. Accordingly, in one embodiment of the invention, any one or all of the prostaglandin, PDE inhibitor and antigen or derivative thereof may be administered at a site distant from the site of disease.

Similarly, unlike other forms of treatment of certain autoimmune diseases, the method may be helpful in preventing inflammatory responses before they start. Thus, the method may be useful in treating patients who, for example because of their age or genetic factors, are predisposed to an autoimmune disease before any inflammatory symptoms show.

The invention also includes inducing tolerance to an antigen in a patient for inhibiting or dampening an immune or inflammatory response in the patient. By “inhibition or dampening” we include increasing the level of IL-10, and/or decreasing the level of IL-12 which leads to an increase in the Th2 response, a decrease in the Th1 response, or an increase in T regulatory cells.

Whether or not a particular patient is one who is expected to benefit from treatment may be determined by the physician.

An effect of the treatment of a patient with a prostaglandin or agonist thereof, a PDE inhibitor and an antigen or a derivative thereof may be the facilitation or improvement of tolerance to that antigen. The antigen may be one which is foreign to the patient, such as an antigen which is involved in irritable bowel syndrome.

It will also be appreciated that the induction of tolerance to an antigen in a patient upon administration of prostaglandin or agonist thereof, PDE inhibitor and the antigen or a derivative thereof, may lead to antigen-specific immune suppression. Thus, the invention includes a method of inducing tolerance to an antigen in a patient to create a state of antigen-specific immune suppression in the patient, the method comprising administering to the patient a prostaglandin or agonist thereof, a PDE inhibitor and the specific antigen or a derivative thereof. Such a state of antigen-specific immune suppression is characterised by raising the threshold of a cell-mediated immune response to stimulus by the specific antigen.

Thus, it will be seen that the invention also provides the use of the combination of a prostaglandin or agonist thereof, a PDE inhibitor and an antigen or a derivative thereof, as an immunosuppressant for that antigen.

The prostaglandin or agonist thereof, the PDE inhibitor, and the antigen or derivative thereof may be administered in any order. Preferably, they are co-administered. However, they may be administered so that the PDE inhibitor can take effect in the accessory cells prior to administration of the prostaglandin or agonist thereof and antigen. The prostaglandin or agonist thereof and the PDE inhibitor may be administered substantially simultaneously, for example in the same composition, with the antigen administered separately. The antigen or derivative thereof may be administered before, after or substantially simultaneously with the prostaglandin or agonist thereof and the PDE inhibitor. The order and timing of administration may be determined by the physician using knowledge of the properties of the antigen, prostaglandin and PDE inhibitor. For example, the prostaglandin (such as misoprostol) may be active over a period of 4 hours following administration. The PDE inhibitor may take of the order of 30 minutes to take effect after administration. Thus, suitable timings of administration can readily be devised from this information.

Where the tolerance to an antigen is desired to be localised to a particular organ, for example to the skin or the bronchial tree and lungs, it is preferred if the prostaglandin or agonist thereof and/or the antigen or derivative thereof is administered locally at the site of the condition. The prostaglandin or agonist thereof may be administered as a gel or cream or vapour or spray or in a “patch” in the case of a condition localised to the skin, or as an inhaled vapour or spray where the site is the lungs or bronchial tree.

The prostaglandin or agonist thereof and/or the antigen or derivative thereof may be administered systemically, such as orally. For example, antigens presented locally to the mucosal immune system, eg via a suppository, are expected to act at mucosal sites remote from the site of administration.

The invention includes the administration of prostaglandin, phosphodiesterase inhibitor and an antigen or derivative thereof to a mucosal site remote from the site of inflammation eg they could be co-administered as a suppository in the case of arthritis. This embodiment may be particularly advantageous as pathologic changes in the gastrointestinal tract may be associated with clinical complaints in multiple organs, including the musculoskeletal system (Alghafeer & Sigal, Bulletin on the Rheumatic Diseases, 51(2): http://www.arthritis.org/research/bulletin/vol51 no2/51_{—}2_printable.asp, incorporated herein by reference). Some reactive arthritis can be triggered by inflammatory bowel diseases, and lymphocytes from the gut mucosa have been reported to migrate to joint tissue in enteropathic arthritis (Salmi & Jalkanen (2001) J. Immunol., 166(7): 4650-7, incorporated herein by reference).

Thus, it is appreciated that the antigen or derivative thereof may be administered to a patient by a variety of means. For example, it can be administered via a mucosal surface of the patient, such as the rectal mucosal surfaces, eg as a suppository; it may be administered via the vagina eg in a pessary; it may be administered via the skin, eg as a gel of cream or patch; it may be administered to the lungs, eg as an aerosol (typically for lung disorders); or orally, eg as a tablet or capsule, (usually for delayed release in the gut).

Without being bound by theory, it is believed that the antigen or derivative thereof is transported to lymphoid tissues such as the lymph nodes in the lymph system or Peyer's patches in the submucosa of the small intestine. Thus, any form of delivery to these tissues is contemplated. The antigen or derivative thereof is presented to circulating T cells by an APC in a tolerising environment of raised IL-10 and lowered IL-12 created by the prostaglandin and the PDE inhibitor. Furthermore, administration of the prostaglandin and PDE inhibitor increases the likelihood that the circulating T cells are regulatory T cells.

The PDE inhibitor may be administered by any suitable route. The PDE inhibitor may reach the desired site of inhibition of PDE, which is typically the leukocytes in relation to the present invention using many different routes of administration. Typically, in one embodiment, the PDE inhibitor is administered systemically. Suitable forms of systemic administration include oral, transcutaneous or by suppository. Many PDE inhibitors are orally available, so it may be convenient to administer the PDE inhibitor orally.

It is also convenient to administer the PDE inhibitor locally. Thus, the PDE inhibitor may be delivered locally, such as on the skin, using, for example, a gel or cream or vapour or spray or in a “patch” as described above in relation to the administration of the prostaglandin or agonist thereof. Similarly, in the case of administration to the bronchial tree or lungs it may be administered as a spray or vapour.

In preferred embodiments of the invention, the prostaglandin or agonist thereof, the PDE inhibitor and the antigen or a derivative thereof may be combined in the same formulation for delivery simultaneously. Thus, the prostaglandin or agonist thereof and the PDE inhibitor may be combined in a gel or a cream or a vapour or spray or “patch” or suppository and administered together to the patient.

Preferably, a suppository has an enteric coating which only releases the active agents in the bowel when the pH has risen. This sort of preparation has been successful in the delivery of glucocorticoids to the bowel (data sheet for Entocort CR).

Alternatively, the prostaglandin or agonist thereof and/or the PDE inhibitor and/or the antigen or derivative thereof, may be administered in a capsule or other suitable form that is swallowed. The capsule or other suitable form has an enteric coating which is pH sensitive, leading to release at an appropriate point in the gastrointestinal tract where it is desired to do so, typically the distal ileum or colon.

Alternatively, the prostaglandin or agonist thereof and/or the PDE inhibitor and/or the antigen or derivative thereof, may be administered directly to the colon or distal ileum using a non-soluble tube or pipe system, such as produced by Egalet.

It is appreciated that the prostaglandin or agonist thereof and/or the PDE inhibitor and/or the antigen may be administered at the same or different sites, and by the same or different modes of administration.

The prostaglandin or agonist thereof may be any suitable prostaglandin or agonist thereof. By “prostaglandin or agonist” we mean any compound which acts as a prostaglandin agonist on a prostaglandin receptor. The prostaglandin agonist need not be a prostanoid. Typically, the agonist is one which binds the EP2 or EP4 receptor. It is preferred that the prostaglandin or agonist thereof is one which is able to stimulate cAMP production in macrophages. It is preferred that the prostaglandin is a PGE or a PGI. Preferably, the prostaglandin is not a PGF or agonist thereof. It is preferred that the prostaglandin or agonist thereof is PGE₂ or a synthetic analogue thereof. Synthetic analogues include those modified at position 15 or 16 by the addition of a methyl group or those where the hydroxyl has been transposed from position 15 to position 16. Preferred examples of analogues of prostaglandin include Butaprost (an EP2 receptor agonist) and 11-deoxy PGE1 (an EP4 receptor agonist). For the avoidance of doubt, the term “prostaglandin” includes naturally-occurring prostaglandins as well as synthetic prostaglandin analogues.

Suitable prostaglandins or agonists thereof include dinoprostone (sold as Propess by Ferring in Europe and Forest in the USA; sold as Prostin E2 by Pharmacia), gemeprost (sold by Farillon), misoprostol (which is sold as Cytotec by Searle and Pharmacia), alprostadil (which is sold as Caverject by Pharmacia and Viridal by Schwarz and MUSE by AstraZeneca) and limaprost.

Misoprostol is a PGE analogue which has EP2 and EP3 agonist effects. Its chemical structure is (±) methyl 11α, 16-dihydroxy-16-methyl-9-oxoprost-13-enoate.

An example of a non-prostanoid compound which acts as a prostaglandin agonist is AH23848, an EP4 receptor agonist.

EP2 agonists which may be useful in the practise of the invention include AH13205.

Suitable prostaglandins also include 19-hydroxy PGE1 and 19-hydroxy PGE2. Prostaglandin agonists are described in EP 1 097 922 and EP 1 114 816, incorporated herein by reference.

Suitable prostaglandins or agonists thereof may also include any of the 19-hydroxy prostaglandin analogues described in U.S. Pat. No. 4,127,612, incorporated herein by reference.

It is preferred that the prostaglandin is prostaglandin E₂ (PGE2). Prostaglandins and agonists thereof, including PGE₂, are commercially available, for example from Pharmacia and Upjohn as Prostin E2.

The PDE inhibitor may be any suitable PDE inhibitor. Preferably, the PDE inhibitor is one which inhibits a PDE which is active in cAMP breakdown. The PDEs which are known to be active in cAMP breakdown are those of the types IV, VII and VIII. Preferably, the PDE inhibitors are selective for type IV or VII or VIII.

Most preferably, the PDE inhibitors are selective for type IV PDE. By “selective” we mean that the inhibitor inhibits the particular type of PDE inhibitor for which it is selective, more potently than another type. Preferably, the type IV selective inhibitor is at least 2 times more potent an inhibitor of type IV PDE than another PDE type. More preferably, the type IV selective inhibitor is at least 5 times, 10 times, 20 times, 30, times 40 times, 50 times, 100 times, 200 times, 500 times or 1000 times more potent an inhibitor of type IV PDE than another PDE type.

Typically, the selective inhibitor is around 5 to 50 times more potent an inhibitor of the selected PDE type than another PDE type. Typically, the selective inhibitor is 5 to 50 times more potent an inhibitor of the selected PDE type than an inhibitor that is considered to be non-selective such as theophylline. Thus, theophylline is 30 times less effective than rolipram.

Preferably, selective inhibition is determined by a comparison of IC₅₀ levels (Dousa (1999) Kidney International 55: 29-62).

Non-specific PDE inhibitors include caffeine, theophylline, 3-isobutyl-1-methylxanthine (IBMX) and pentoxifylline (3,7-dihydro-3,7-dimethyl-1-(5-oxohexyl)-1H-purine-2,6-dione), although caffeine is not as active as the others and so is less preferred. The IC₅₀ value for IBMX is 2-50 μM.

U.S. Pat. No. 6,127,378, incorporated herein by reference, discloses phenanthridines substituted in the 6 position that are described as selective PDE inhibitors (mainly of type IV), that may be suitable for use in the methods of the invention.

Specific (or selective) type IV PDE inhibitors include rolipram (4-[3-cyclopentyloxy-4-methoxyphenyl]-2-pyrrolidinone) and Ro-20-1724 (4-[3-butoxy-4-methoxybenzyl]-2-imidazolidinone). The IC₅₀ for rolipram is 800 nM, and the IC₅₀ for Ro-20-1724 is 2 μM.

Another suitable PDE type IV selective inhibitor is denbufylline (1,3-di-n-butyl-7-(2-oxopropyl)-xanthine).

CP 80 633 (Hanifin et al (1996) J. Invest. Dermatol. 107, 51-56), CP 102 995 and CP 76 593 are also all potent type IV inhibitors (available from Central Research Division, Pfizer Inc. Groton, Conn.).

Other high affinity type IV selective PDE inhibitors include CPD 840, RP 73401, and RS 33793 (Dousa, 1999). The high affinity type IV selective PDE inhibitors have a Kᵢ of approximately 1 nM while the lower affinity inhibitors have a Kᵢ of about 1 μM.

The disclosures in Dousa (1999); Müller et al (1996, Trends Pharmacol. Sci. 17: 294-298); Palfreyman & Souness (1996, Prog Med Chem 33: 1-52); Stafford & Feldman (1996, Annual Reports in Medicinal Chemistry (vol 31) pp 71-80; Ed. Bristol, Academic Press, NY, USA); and Teixeira et al (1997, Trends Pharmacol. Sci. 18: 164-171) relating to type IV PDE selective inhibitors are incorporated herein by reference.

Typically, when a type IV PDE-selective inhibitor is administered orally, around 1 to 30 mg is used. Thus, a typical oral dose of rolipram or denbufylline is 1 mg or 5 mg or 10 mg or 30 mg. When a non-selective PDE inhibitor is used, such as theophylline, and it is administered orally, the dose is between 5 and 50 mg, such as 5 or 10 or 20 or 30 or 40 or 50 mg.

In one embodiment the prostaglandin or agonist thereof is administered orally. In particular the prostaglandin or agonist thereof is a prostaglandin analogue which has been modified to reduce its catabolism and which is orally available (such as misoprostol).

Although the PDE inhibitor can be administered by any suitable means and by any suitable route when the prostaglandin or agonist thereof is administered orally, it is preferred that the PDE inhibitor is also administered orally. It is also preferred if the prostaglandin or agonist thereof and PDE inhibitor are administered simultaneously, for example in the same composition. More preferably, the prostaglandin or agonist thereof, the PDE inhibitor and the antigen are administered simultaneously, for example in the same composition.

Thus, in a preferred embodiment, the method of the invention makes use of the oral administration of a prostaglandin analogue which has been modified to reduce its catabolism and which is orally available (such as misoprostol) and the oral administration of a PDE inhibitor, such as rolipram. Typically, the antigen or derivative thereof is also administered orally. The advantages of oral administration is that it generally has good compliance compared to other modes of administration.

The inventor believes that the combination of PDE inhibitor with the orally available prostaglandin or agonist thereof will mean that a lower dose of oral prostaglandin will be required than in the absence of PDE inhibitor. It is believed by the inventor that this will have the advantage of reducing side effects caused by the oral prostaglandin or agonist thereof, such as muscle cramps.

The data described in the Figures and Examples shows that typically a higher concentration of 19-hydroxy PGE would be necessary to achieve similar effects to PGE. However, 19 hydroxy PGE has the advantage of being more rapidly catabolised.

Thus, preferably, the combination of a PDE inhibitor and prostaglandin or agonist thereof, comprises a selective type IV PDE inhibitor and a 19-hydroxy PGE. Typically, 0.1-100 μg of 19 hydroxy PGE and 1-250 μg Rolipram in 5 ml saline would be administered.

Typically, 100 to 800 μg of misoprostol is administered orally daily with 1 to 30 mg of rolipram or denbufylline.

As described above, the prostaglandin or agonist thereof can be used orally in combination with a PDE inhibitor at a lower dose than in the absence of PDE inhibitor.

Typically, the dose of PDE inhibitor is as described above and the prostaglandin, such as misoprostol, is administered at a dose of 100 to 400 μg.

Typically, the antigen or derivative thereof is administered in a dose between about 100 ng and about 100 mg, more typically about 100 μg.

A second aspect of the invention provides the use of a prostaglandin or agonist thereof in the manufacture of a medicament for inducing tolerance to an antigen in a patient wherein the patient is administered a PDE inhibitor and the antigen or a derivative thereof. Thus, the patient may already have been administered the PDE inhibitor and the antigen or derivative thereof before administration of the prostaglandin or agonist thereof, or is administered the PDE inhibitor and the antigen or derivative thereof at the same time as the prostaglandin or agonist thereof, or will be administered the PDE inhibitor and the antigen or derivative thereof after administration of the prostaglandin or agonist thereof.

A third aspect of the invention is the use of a PDE inhibitor in the manufacture of a medicament for inducing tolerance to an antigen in a patient wherein the patient is administered a prostaglandin or agonist thereof and the antigen or a derivative thereof. Thus, the patient may already have been administered the prostaglandin or agonist thereof and the antigen or derivative thereof before administration of the PDE inhibitor, or is administered the prostaglandin or agonist thereof and the antigen or derivative thereof at the same time as the PDE inhibitor, or will be administered the prostaglandin or agonist thereof and the antigen or derivative thereof after administration of the PDE inhibitor.

A fourth aspect of the invention is the use of an antigen or a derivative thereof in the manufacture of a medicament for inducing tolerance to the antigen in a patient wherein the patient is administered a prostaglandin or agonist thereof and a PDE inhibitor. Thus, the patient may already have been administered the prostaglandin or agonist thereof and PDE inhibitor before administration of the antigen or derivative thereof, or is administered the prostaglandin or agonist thereof and the PDE inhibitor at the same time as the antigen or derivative thereof, or will be administered the prostaglandin or agonist thereof and the PDE inhibitor after administration of the antigen or derivative thereof.

A fifth aspect of the invention provides the use of a prostaglandin or agonist thereof and PDE inhibitor in the manufacture of a medicament for inducing tolerance to an antigen in a patient wherein the patient is administered the antigen or a derivative thereof. Thus, the patient may already have been administered the antigen or derivative thereof before administration of the prostaglandin or agonist thereof and PDE inhibitor; or is administered the antigen or derivative thereof at the same time as the prostaglandin or agonist thereof and PDE inhibitor, or will be administered the antigen or derivative thereof after administration of the prostaglandin or agonist thereof and PDE inhibitor.

A sixth aspect of the invention is the use of a PDE inhibitor and an antigen or a derivative thereof in the manufacture of a medicament for inducing tolerance to the antigen in a patient wherein the patient is administered a prostaglandin or agonist thereof. Thus, the patient may already have been administered the prostaglandin or agonist thereof before administration of the PDE inhibitor and the antigen or derivative thereof, or is administered the prostaglandin or agonist thereof at the same time as the PDE inhibitor and the antigen or derivative thereof, or will be administered the prostaglandin or agonist thereof after administration of the PDE inhibitor and the antigen or derivative thereof.

A seventh aspect of the invention provides the use of a prostaglandin or agonist thereof, and an antigen or a derivative thereof, in the manufacture of a medicament for inducing tolerance to the antigen in a patient wherein the patient is administered a PDE inhibitor. Thus, the patient may already have been administered the PDE inhibitor before administration of the prostaglandin or agonist thereof and the antigen or derivative thereof, or is administered the PDE inhibitor at the same time as the prostaglandin or agonist thereof and the antigen or derivative thereof, or will be administered the PDE inhibitor after administration of the prostaglandin or agonist thereof and the antigen or derivative thereof.

An eighth aspect of the invention provides the use of a combination of a prostaglandin or agonist thereof, a PDE inhibitor and an antigen or a derivative thereof in the manufacture of a medicament for inducing tolerance to the antigen in a patient. Thus, the prostaglandin or agonist thereof, PDE inhibitor and the antigen or a erivative thereof may be combined in the same medicament before administration to the patient.

Preferably, the use according to the second, third and fourth, fifth, sixth, seventh and eighth aspects is in treating an aberrant or undesired immune or inflammatory response in the patient.

The preferences for the prostaglandin or agonist thereof, PDE inhibitors, antigens, routes of administration, doses and so on for the second, third, fourth, fifth, sixth, seventh and eighth aspects of the invention are the same as for the first aspect of the invention.

A ninth aspect of the invention provides a therapeutic system for inducing tolerance to an antigen, the system comprising a prostaglandin or agonist thereof, a PDE inhibitor and the antigen or a derivative thereof. The therapeutic system may also be termed a “kit of parts”.

Preferably, the therapeutic system contains a preferred prostaglandin or agonist thereof as defined in the first aspect of the invention. Still preferably, the therapeutic system contains a preferred PDE inhibitor as defined in the first aspect of the invention. Still preferably, the therapeutic system contains a preferred antigen, or derivative thereof, as defined in the first aspect of the invention. The therapeutic system or kit of parts may suitably contain the prostaglandin or agonist thereof, the PDE inhibitor, and the antigen or derivative thereof packaged and presented in suitable formulations for use in combination, either for administration simultaneously or for administration which is separated in time. Thus, for example, in one embodiment where the prostaglandin or agonist thereof, PDE inhibitor and antigen or derivative thereof are for simultaneous administration locally to the skin, the therapeutic system may contain a gel or cream or spray or vapour or “patch” which contains a combination of prostaglandin or agonist thereof PDE inhibitor and the antigen or derivative thereof. Alternatively, in another embodiment where the prostaglandin or agonist thereof, PDE inhibitor and antigen or derivative thereof are for separate administration in a particular treatment regime, they are packaged or formulated separately. For example, the prostaglandin or agonist thereof may be formulated for administration locally using a cream or gel or spray or vapour or “patch”, and the PDE inhibitor and the antigen or derivative thereof are packaged or formulated for systemic administration such as oral administration.

The formulations of the prostaglandin or agonist thereof alone or PDE inhibitor alone or the antigen or derivative thereof alone, or any combinations thereof may conveniently be presented in unit dosage form and may be prepared by any of the methods well known in the art of pharmacy. Such methods include the step of bringing into association the active ingredients used in the invention with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention suitable for oral administration (eg of the PDE inhibitor or of a suitable prostaglandin or agonist thereof or antigen) may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

For local administration to the skin, it may be convenient to formulate the prostaglandin or agonist thereof and/or PDE inhibitor and/or antigen or derivative thereof in combination with a dispersion agent or an agent which allows for increased transdermal or transmucosal transfer or penetration, such a dimethyl sulphoxide (DMSO) and the like. Suitable agents are ones which are compatible with the prostaglandin or agonist thereof and/or PDE inhibitor (eg are solvents thereof).

A tenth aspect of the invention provides a composition comprising a prostaglandin or agonist thereof, a PDE inhibitor, and an antigen to which it is desired to induce tolerance in a patient or a derivative thereof. Before the present invention, there has been no reason to combine in a composition a prostaglandin or agonist thereof, a PDE inhibitor and an antigen to which it is desired to induce tolerance or a derivative thereof. Preferably, the prostaglandin or agonist thereof is one which is preferred in the first aspect of the invention. Also preferably, the PDE inhibitor is one which is preferred in the first aspect of the invention. Also preferably, the antigen or derivative thereof is one which is preferred in the first aspect of the invention.

The composition may be used in the practice of certain embodiments of the first aspect of the invention. Thus, an eleventh aspect of the invention provides a composition comprising a prostaglandin or agonist thereof, a PDE inhibitor, and an antigen to which it is desired to induce tolerance in a patient, or a derivative thereof, for use in medicine. The composition is therefore packaged and presented for use in medicine. The composition may be used in human or veterinary medicine; preferably, it is used in human medicine.

Typically, the composition further comprises a pharmaceutically acceptable carrier. Thus, a twelfth aspect of the invention provides a pharmaceutical composition (or formulation as it may be termed) comprising a prostaglandin or agonist thereof, a PDE inhibitor, an antigen to which it is desired to induce tolerance in a patient or a derivative thereof, and a pharmaceutically acceptable carrier. The carrier(s) must be “acceptable” in the sense of being compatible with the composition of the invention and not deleterious to the recipients thereof. Typically, the carriers will be water or saline which will be sterile and pyrogen free.

The patient on which the method or medicament is used is preferably a human although the patient may be any mammal such as a cat, dog, horse, cow, sheep, horse, pig and so on.

It will be appreciated that the method or medicament may be used before symptoms indicating a need to induce tolerance of an antigen becomes apparent in the patient to be treated, or, either alternatively or in addition, the using of the method or medicament may be used after symptoms or signs become apparent. Thus, in the case of a patient receiving an organ or tissue transplant, it may be beneficial to administer the prostaglandin or agonist thereof, PDE inhibitor and antigen or derivative thereof before the transplantation surgery is started. It may be further beneficial to continue the administration during or after completion of the transplant or graft surgery. The necessary dosage may be determined by the physician, according to the degree of tolerance that is required.

It will further be appreciated that each of the prostaglandin or agonist thereof, the PDE inhibitor, and the antigen or derivative thereof, may be administered as a single dose, or in multiple smaller doses which achieve the same therapeutic effect. The frequency of administration may vary according to the convenience of the physician administering the dose or the patient.

It is appreciated that it may be preferable to minimise the exposure of the patient to other antigens other than the one to which it is desired to induce tolerance. In some cases, this may include keeping the patient in an isolation “bubble” as is known in the art for immunosuppressed patients.

It is appreciated that tolerance to more than one antigen may be desired. Therefore reference to methods, uses and compositions comprising an antigen to which it is desired to induce tolerance, may include two or three or four or five or more antigens to which it is desired to induce tolerance.

Pregnancy is likely to be a contraindication for the present invention. In fact, pregnancy is a contraindication for several prostaglandins including misoprostol. Cytotec (misoprostol) does not cause hypotension, but this may be a possible risk with the method of the invention.

The invention will now be described in more detail with the aid of the following Figures and Examples.

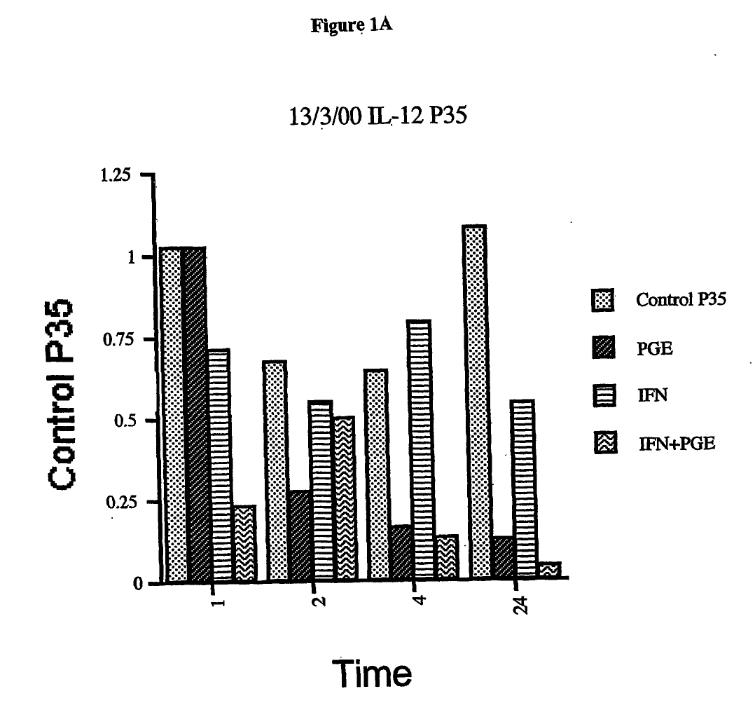

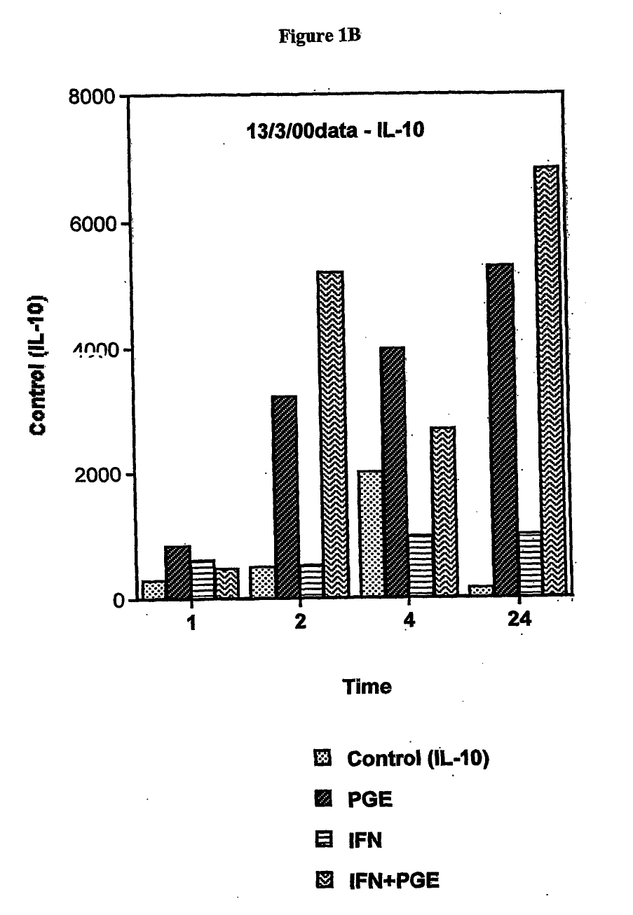

FIG. 1

Expression of mRNA for cytokines IL-10 and IL-12 subunit p35. Experiment carried out on U937 cells (pro-monocytes) in the presence of Rolipram at 1 μg/ml=4 μM and indomethacin 10 μM. The indomethacin prevents prostaglandin synthesis from cells. Note that the effect of PGE+Rolipram is a marked stimulation of IL-10 and an inhibition of IL-12 both for unstimulated and IFNγ stimulated cells. Vertical scale is a measure of mRNA compared to a control sample as measured by real-time quantitative PCR (Taqman).

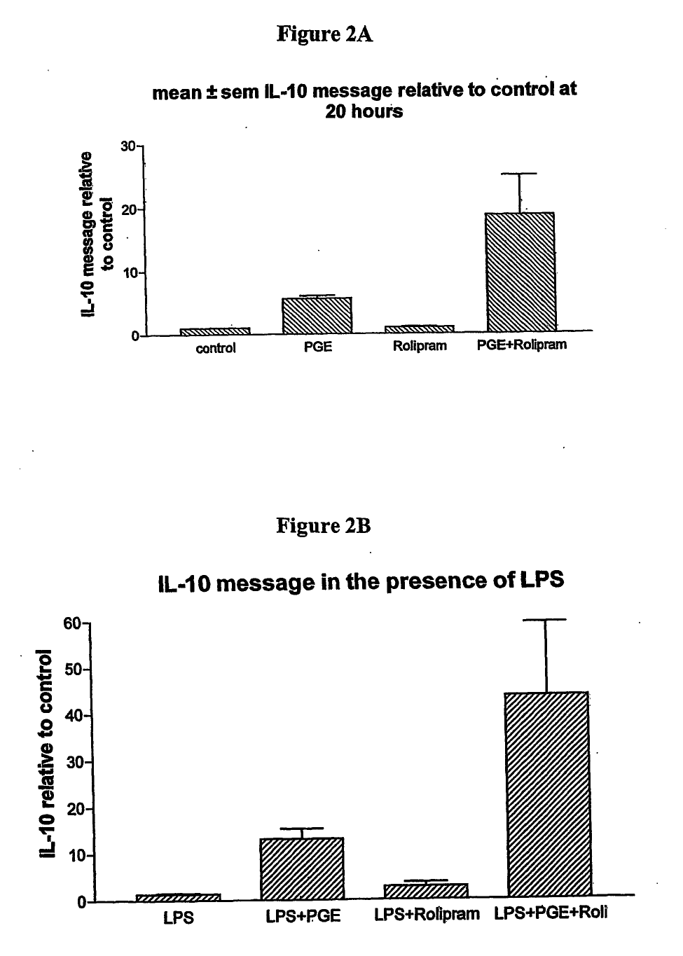

FIG. 2

FIG. 2A is a graph showing the effect of PGE and Rolipram on the production of IL-10 mRNA in U937 cells. FIG. 2B is a graph showing the effect of LPS, PGE and Rolipram on the production of IL-10 mRNA in U937 cells. FIG. 2C is a graph showing the effect of LPS, PGE and Rolipram on IL-10 release from U937 cells. FIG. 2D is a graph showing the effect of PGE and Rolipram on IL-10 release from U937 cells.

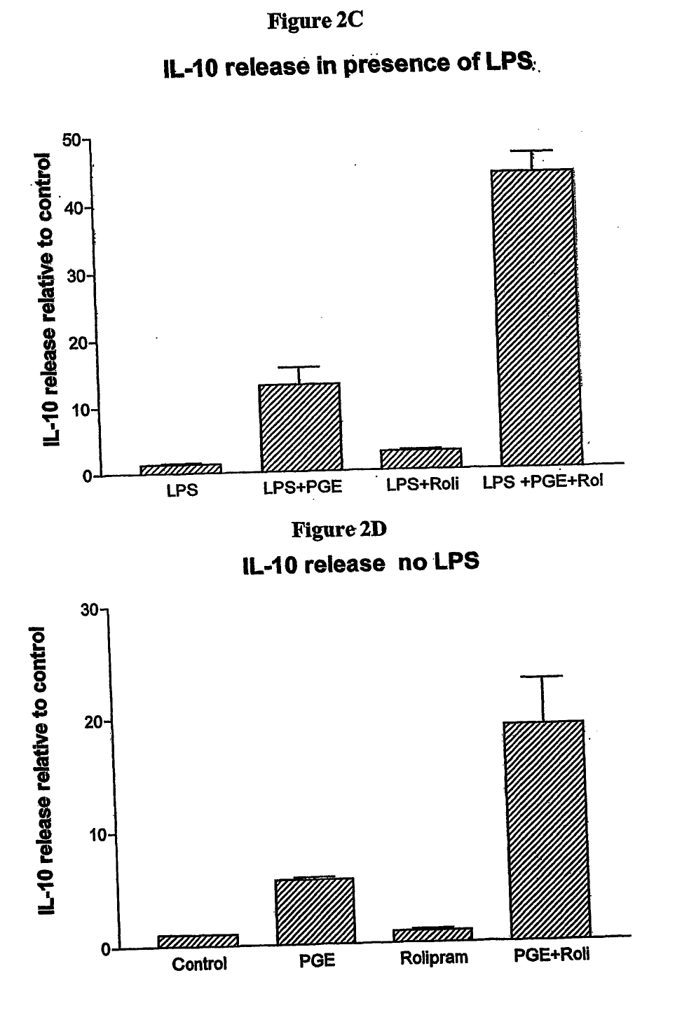

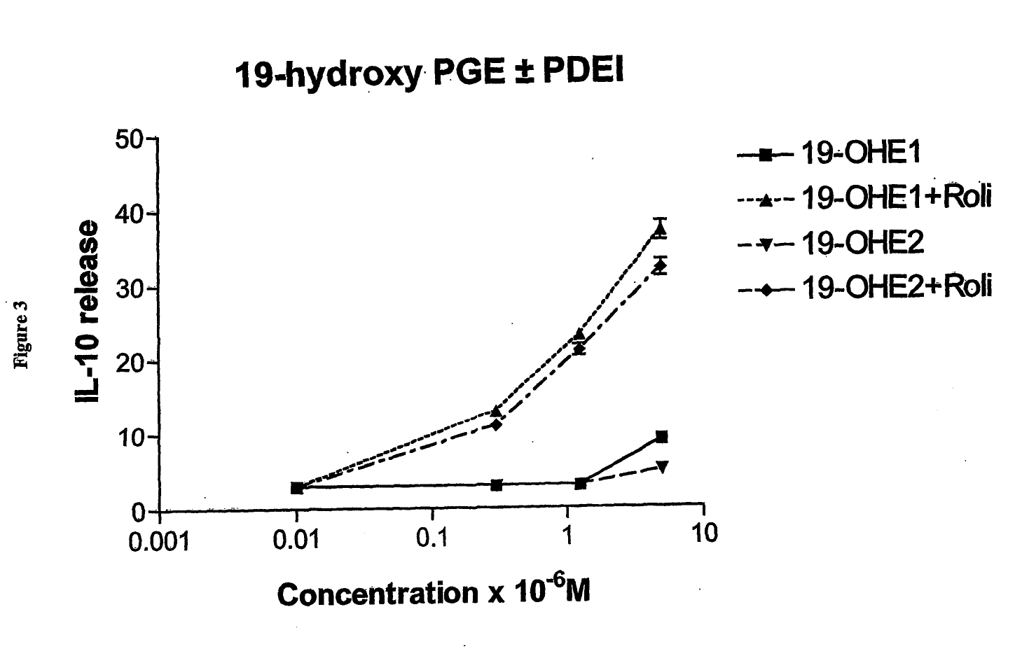

FIG. 3

A graph showing the effect of 19 hydroxy PGE1 and 19 hydroxy PGE2 on the stimulation of IL-10 in the presence and absence of rolipram.

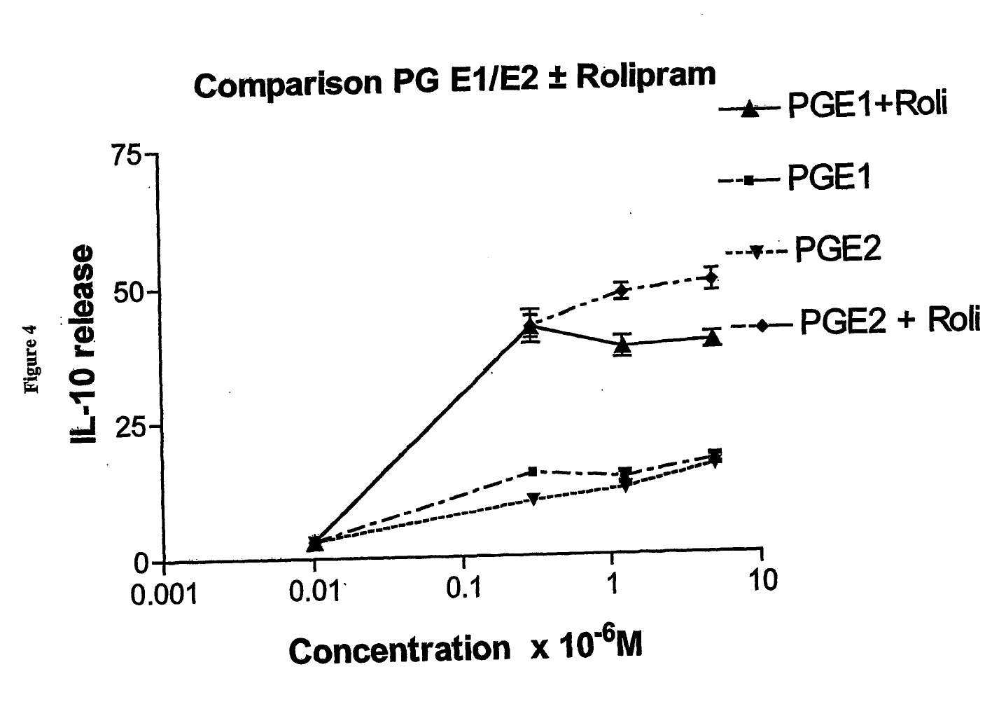

FIG. 4

A graph showing the effect of PGE1 and PGE2 on the stimulation of IL-10 in the presence and absence of rolipram.

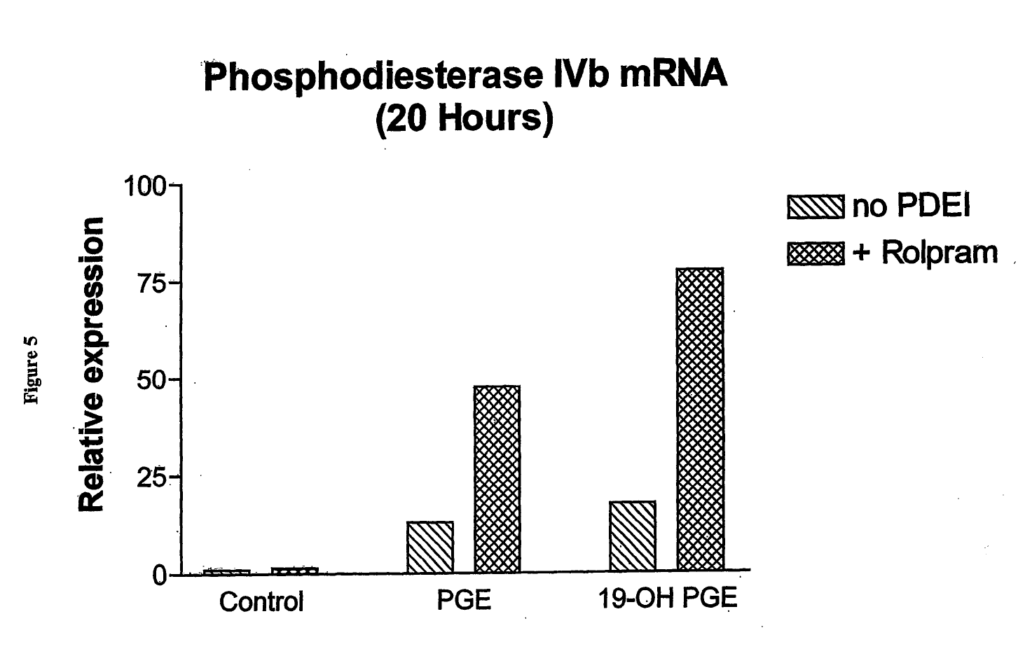

FIG. 5

A graph showing the effect of PGE and 19 hydroxy PGE on the production of phosphodiesterase IV b mRNA in the presence and absence of rolipram.

### EXAMPLE 1

### Effect of the Combination of PGE and Rolipram on IL-10 and IL-12 Production by U-937 (Promonocyte) Cells

U 937 (human monocyte cell line) cells were grown in RPMI (PAA Laboratories) medium with 10% fetal calf serum added (PAA Laboratories). Cells were treated with prostaglandin E 2 at 10⁻6 Molar or with Interferon-γ at 10 ng/ml for 24 hours. Rolipram at 1 μg/ml and indomethacin at 10 μM was present in all wells. Cells were pelleted and the mRNA was extracted with Tri reagent (Sigma, Poole, UK). Total RNA was obtained by addition of chloroform and subsequent isopropanol precipitation. RNA was reverse transcribed with reverse transcriptase (Applied Biosystems) and random hexamers (Applied Biosystems). Probes and primers for IL-10 and IL-12 (p35) were designed using Primer Express (Applied Biosystems) and were as follows:

IL-12 p35 primers
CCACTCCAGACCCAGGAATG
TGTCTGGCCTTCTGGAGCAT
IL-12 probe
TCCCATGCCTTCACCACTCCCAA
IL-10 primers
CTACGGCGCTGTCATCGAT
TGGAGCTTATTAAAGGCATTCTTCA
IL-10 probe
CTTCCCTGTGAAAACAAGAGCAAGGCC

Template was amplified in a Taqman 7700 machine for 40 cycles using FAM/TAMRA dyes on the probe. The Applied Biosystems Kit was used to amplify and detect ribosomal (18S)RNA as a control. After 40 cycles the Ct (related to cycle number at which signal appears) for the FAM and the 18S (VIC) were recorded and absolute relative quantitation was achieved using the formula 2^{-ΔΔ}^{^{Ct}}.

The results of this experiment are described in the legend to FIG. 1. They show that there is a synergistic between a prostaglandin (PGE2) and a PDE inhibitor (rolipram) on the release of IL-10 from cells of the immune system and that there is a marked stimulation of IL-10 and inhibition of IL-12 in cells of the immune system when a prostaglandin (PGE2) and a PDE inhibitor (rolipram) are used in combination.

### EXAMPLE 2

### Stimulation of IL-10 Production is Achieved with or without LPS

U 937 cells were grown in RPMI (PAA Laboratories) medium with 10% fetal calf serum added (PAA Laboratories). 2×10⁶ cells per flask were treated with prostaglandin E₂ at 10⁻⁶ Molar or with Rolipram (4×10⁻⁶) for 24 hours. Medium was removed at 20 hours and analysed by ELISA. A capture antibody (Pharmingen) was coated onto 96 well plates and culture medium was added each well. A standard curve was created with recombinant IL-10 protein. After incubation and washing, a biotin labelled monoclonal antibody (Pharmingen) was added and following incubation and washing, peroxidase labelled streptavidin was added. After washing a tetramethyl benzidine substrate was added and colour developed in proportion to IL-10 in the original sample/standard. Colour was read using a plate photometer (Labsystems, Multiskan). Mean concentrations (N=3) in controls with no lipopolysaccharide (LPS) were 38.2 μg/ml and in the presence of LPS (100 nM) they were 43.9 prostaglandin/ml.

After the incubation (20 hours), cells were pelleted and the mRNA was extracted with Tri-reagent (Sigma, Poole, UK). Total RNA was obtained by addition of chloroform and subsequent isopropanol precipitation. RNA was reverse transcribed with reverse transcriptase (Applied Biosystems) and random hexamers (Applied Biosystems). Probes and primers for IL-10 and IL-12 (p35) were designed using Primer Express (Applied Biosystems) and were as follows:

IL-12 p35 primers
CCACTCCAGACCCAGGAATG
TGTCTGGCCTTCTGGAGCAT
IL-12 probe
TCCCATGCCTTCACCACTCCCAA
IL-10 primers
CTACGGCGCTGTCATCGAT
TGGAGCTTATTAAAGGCATTCTTCA
IL-10 probe
CTTCCCTGTGAAAACAAGAGCAAGGCC

Template was amplified in a Taqman 7700 machine for 40 cycles using FAM/TAMRA dyes on the probe. The Applied Biosystems kit was used to amplify and detect ribosomal (18S)RNA (using VIC/TAMRA dyes) as an internal control in the same reaction tube. After 40 cycles the Ct (related to cycle number at which signal appears) for the FAM and the 18S (VIC) were recorded and absolute relative quantitation was achieved using the formula 2^{-ΔΔCt} where Δ refers to the difference between the FAM and VIC signal related to an standard comparator included in each run.

### EXAMPLE 3

The effect of PGE1, PGE2, 19 hydroxy PGE1 and 19 hydroxy PGE2 on the stimulation of IL-10 in the presence and absence of rolipram was investigated as described above in Example 2. IL-10 levels were measured using an ELISA assay (R&D Ltd, Oxford). Measurement was performed according to the manufacturer's instructions. Results are shown in FIGS. 3 and 4.

### EXAMPLE 4

The mRNA for phosphodiesterase IV-b was measured as described in Example 2 above. mRNA was extracted after four hours of incubation. The concentration of the PGE was 1×10⁻⁶ and that of the 19-hydroxy PGE₂ was 5×10⁻⁶. The following primers and Taqman probe were used for quantitation of PDE IV b mRNA.

Forward
CCTTCAGTAGCACCGGAATCA
Reverse
CAAACAAACACACAGGCATGTAGTT
Probe
AGCCTGCAGCCGCTCCAGCC

Results are shown in FIG. 5. An increase in PDE activity follows both PGE and 19-hydroxy PGE application, which appears to be a direct negative feedback to reduce the effect of the stimulus. Use of a PGE and a type IV selective PDE inhibitor increases PDE message levels even further, but then the synthesised phosphodiesterase is nullified by the presence of the inhibitor.

### EXAMPLE 5

### Treatment of Demyelinating Disease

A patient with demyelinating disease is administered 800 μg misoprostol and 25 mg rolipram orally, daily together with myelin.

### EXAMPLE 6

### Treatment of Rheumatoid Arthritis

A patient with rheumatoid arthritis is administered 800 μg misoprostol and 25 mg rolipram orally, daily together with type II collagen.

### EXAMPLE 7

### Treatment of Demyelinating Disease

A patient with demyelinating disease is administered 200 μg misoprostol and 5 mg rolipram by suppository, daily together with 200 μg myelin.

### EXAMPLE 8

### Treatment of Rheumatoid Arthritis

A patient with rheumatoid arthritis is administered 200 μg misoprostol and 5 mg rolipram by suppository, daily together with 200 μg type II collagen.

1. A method of inducing tolerance to an antigen in a patient, the method comprising administering to the patient a prostaglandin or agonist thereof, a phosphodiesterase (PDE) inhibitor, and the antigen or derivative thereof.

2. A method according to claim 1 wherein the prostaglandin or agonist thereof and/or the PDE inhibitor and/or the antigen or derivative thereof is administered locally at a site where tolerance is required.

3. A method according to claim 1 or wherein the prostaglandin or agonist thereof and/or PDE inhibitor and/or the antigen or derivative thereof is administered systemically.

4. A method according to claim 3 wherein the prostaglandin or agonist thereof and/or PDE inhibitor and/or the antigen or derivative thereof is administered orally.

5. A method according to claim 3 wherein the prostaglandin or agonist thereof and/or PDE inhibitor and/or the antigen or derivative thereof is administered as a suppository or capsule.

6. A method according to claim 5 wherein the suppository or capsule has an enteric coating for release of the prostaglandin or agonist thereof and/or PDE inhibitor and/or the antigen or derivative thereof in the bowel of the patient.

7. A method according to claim 1 wherein any two of the prostaglandin or agonist thereof, the PDE inhibitor, and the antigen or derivative thereof are administered simultaneously.

8. A method according to claim 1 wherein the prostaglandin or agonist thereof, the PDE inhibitor, and the antigen or derivative thereof are administered simultaneously.

9. A method according to claim 1 wherein the prostaglandin or agonist thereof is any one of a prostaglandin E, prostaglandin E₂ or an analogue thereof, dinoprostone, gemeprost, misoprostol, alprostadil, limaprost, butaprost, 11-deoxy PGE1, AH23848, AH13205, 19-hydroxy PGE1 or 19-hydroxy PGE2.

10. A method according to claim 1 wherein the prostaglandin is a 19-hydroxy PGE.

11. A method according to claim 1 wherein the PDE inhibitor is selective for any of type IV, VII or VIII PDE.

12. A method according to claim 1 wherein the PDE inhibitor is any one of 3-isobutyl-1-methylxanthine (IBMX), pentoxifylline (3,7-dihydro-3,7-dimethyl-1-(5-oxohexyl)-1H-purine-2,6-dione), rolipram (4-[3-cyclopentyloxy-4-methoxyphenyl]-2-pyrrolidinone), CP80 633, CP102 995, CP76 593, Ro-20-1724 (4-[3-butoxy-4-methoxybenzyl]-2-imidazolidinone), theophylline, or denbufylline (1,3-di-n-butyl-7-(2-oxopropyl)-xanthine).

13. A method according to claim 1 wherein the PDE inhibitor is selective for type IV PDE.

14. A method according to claim 13 wherein the PDE inhibitor selective for type IV PDE is any one of rolipram (4-[3-cyclopentyloxy-4-methoxyphenyl]-2-pyrrolidinone), CP80 633, CP102 995, CP76 593, Ro-20-1724 (4-[3-butoxy-4-methoxybenzyl]-2-imidazolidinone), denbufylline (1,3-di-n-butyl-7-(2-oxopropyl)-xanthine), or CDP840, RP73401 or RS33793.

15. A method of combating a disease or condition associated with transplant rejection comprising:
performing the method according to claim 1 wherein said administering is effective to combat a disease or condition associated with transplant rejection.

16. A method according to claim 15 wherein the disease or condition associated with transplant rejection comprises graft versus host disease or host versus graft disease.

17. A method according to claim 15 wherein the prostaglandin or agonist thereof and/or PDE inhibitor and/or antigen or derivative thereof are administered prior to the transplant.

18. A method according to claim 15 wherein the antigen is HLA-A2.

19. A method for treating an autoimmune disease or condition comprising:
performing the method according to claim 1, wherein said administering is effective to treat an autoimmune disease or condition.

20. A method according to claim 19 wherein the antigen is a self-antigen.

21. A method according to claim 19, wherein the autoimmune disease is pernicious anaemia, and the antigen is vitamin B₁₂; the disease is Addison's disease, and the antigen is adrenal antigen; if the disease is IDDM, and the antigen is glutamic acid decarboxylase (GAD), insulin, or IA-2; the disease is Goodpasture's syndrome or renal vasculitis, and the antigen is renal antigen or endothelial antigen; the disease is myasthenia gravis, and the antigen is the acetyl choline receptor; the disease is sympathetic ophthalmia, and the antigen is ocular antigen; the disease is multiple sclerosis (MS), and the antigen is myelin basic protein (MBP), proteolipid protein (PLP), or myelin oligodendrocyte glycoprotein (MOG); the disease is autoimmune haemolytic anaemia, and the antigen is red cell antigen; the disease is idiopathic leucopenia, and the antigen is leukocyte antigen; the disease is ulcerative colitis, and the antigen is a food antigen or a viral antigen; the disease is dermatomyositis, and the antigen is smooth muscle antigen; the disease is scleroderma, and the antigen is a connective tissue antigen; the disease is mixed connective tissue disease, and the antigen is a connective tissue antigen; the disease is irritable bowel syndrome, and the antigen is a food antigen; the disease is systemic lupus erythmatosus (SLE), and the antigen is a histone protein or immunoglobulin heavy chain; the disease is Hashimoto's disease, primary myxoedema or thyrotoxicosis, and the antigen is thyroid antigen; the disease is rheumatoid arthritis, and the antigen is type II collagen or a heat shock protein (HSP); the disease is thyroiditis, and the antigen is thyroglobulin; the disease is Behcet's disease, and the antigen is Sag, HLA-B44, B51, or HSP65; the disease is Coeliac disease/Dermatitis herpetiformis, and the antigen is gliadin or the α fraction thereof; or the disease is demyelinating disease, and the antigen is myelin.

22. A method for treating an allergic disease or condition in the patient comprising:
performing the method according to claim 1, wherein said administering is effective to treat an allergic disease or condition in the patient.

23. A method according to claim 22 wherein the allergic disease or condition is allergic asthma.

24. A method according to claim 22, wherein the antigen is a mite allergen, a dust allergen, a cat allergen, a dog allergen or a horse allergen.

25. A method according to claim 1, wherein the induced tolerance to the antigen is effective to treat an aberrant or undesired immune or inflammatory response to the antigen in the patient.

26. A method according to claim 25 wherein the aberrant or undesired immune or inflammatory response involves a deficiency in IL-10 production and/or an increase in IL-12 production.

27. Use of one or more of (i) a prostaglandin or agonist thereof, (ii) a PDE inhibitor, and (iii) an antigen or a derivative thereof in the manufacture of a medicament for inducing tolerance to the an antigen in a patient wherein the patient is administered any of (i), (ii), and (iii) that are not present in the medicament.

28-33. (canceled)

34. Use according to claim 27 wherein the PDE inhibitor is selective for any of type IV, VII or VIII PDE.

35. Use according to claim 27 wherein the PDE inhibitor is any one of IBMX, pentoxifylline, CP80 633, CP102 995, CP76 593, rolipram, Ro-20-1724, theophylline, caffeine or denbufylline.

36. Use according to claim 27 wherein the PDE inhibitor is selective for type IV PDE.

37. Use according to claim 36 wherein the PDE inhibitor selective for type IV PDE is any one of rolipram, CP80 633, CP102 995, CP76 593, Ro-20-1724, denbufylline, CDP840, RP73401, or 7S33793.

38. Use according to claim 27 wherein the prostaglandin or agonist thereof is any one of a prostaglandin E, prostaglandin E₂ or an analogue thereof, carboprost, dinoprostone, gemeprost, misoprostol, alprostadil, lamaprost, butaprost, 11-deoxy PGE1, AH23848, AH13205, 19-hydroxy PGE1 or 19-hydroxy PGE2 or agonist thereof.

39. Use according to claim 27 wherein the prostaglandin is a 19-hydroxy PGE.

40. Use according to claim 27 wherein the medicament is administered locally at a site where tolerance is required.

41. Use according to claim 27 wherein the medicament is formulated to be administered systemically.

42. Use according to claim 41 wherein the medicament is formulated to be administered orally.

43. Use according to claim 41 wherein the medicament is formulated to be administered as a suppository or capsule.

44. Use according to claim 43 wherein the suppository or capsule has an enteric coating for release of the prostaglandin or agonist thereof and/or PDE inhibitor and/or the antigen or derivative thereof in the bowel of the patient.

45. Use according to claim 27 for combating a disease or condition associated with transplant rejection.

46. Use according to claim 45 wherein the disease or condition associated with transplant rejection comprises graft versus host disease or host versus graft disease.

47. Use according to claim 45 wherein the medicament is administered prior to the transplant.

48. Use according to claim 45 wherein the antigen is HLA-A2.

49. Use according to claim 27 for treating an autoimmune disease or condition.

50. Use according to claim 49 wherein the antigen is a self-antigen.

51. Use according to claim 49, wherein the autoimmune disease is pernicious anaemia, and the antigen is vitamin B₁₂; the disease is Addison's disease, and the antigen is adrenal antigen; the disease is IDDM, and the antigen is GAD, insulin, or IA-2; the disease is Goodpasture's syndrome or renal vasculitis, and the antigen is renal antigen or endothelial antigen; the disease is myasthenia gravis, and the antigen is the acetyl choline receptor; the disease is sympathetic ophthalmia, and the antigen is ocular antigen; the disease is MS, and the antigen is MBP, PLP, or MOG; the disease is autoimmune haemolytic anaemia, and the antigen is red cell antigen; the disease is idiopathic leucopenia, and the antigen is leukocyte antigen; the disease is ulcerative colitis, and the antigen is a food antigen or a viral antigen; the disease is dermatomyositis, and the antigen is smooth muscle antigen; the disease is scleroderma, and the antigen is a connective tissue antigen; the disease is mixed connective tissue disease, and the antigen is a connective tissue antigen; the disease is irritable bowel syndrome, and the antigen is a food antigen; the disease is SLE, and the antigen is a histone protein or immunoglobulin heavy chain; the disease is Hashimoto's disease, primary myxoedema or thyrotoxicosis, and the antigen is thyroid antigen; the disease is rheumatoid arthritis, and the antigen is type II collagen or an HSP; the disease is thyroiditis, and the antigen is thyroglobulin; the disease is Behcet's disease, and the antigen is Sag, HLA-B44, B51, or HSP65; the disease is Coeliac disease/Dermatitis herpetiformis, and the antigen is gliadin or the α fraction thereof; or the disease is demyelinating disease, and the antigen is myelin.

52. Use according to claim 27 for treating an allergic disease or condition in the patient.

53. Use according to claim 52 wherein the allergic disease or condition is allergic asthma.

54. Use according to claim 52, wherein antigen is a mite allergen, a dust allergen, a cat allergen, a dog allergen or a horse allergen.

55. Use according to claim 27 wherein the tolerance to the antigen is to treat an aberrant or undesired immune or inflammatory response to the antigen in the patient.

56. Use according to claim 55 wherein the aberrant or undesired immune or inflammatory response involves a deficiency in IL-10 production and/or an increase in IL-12 production.

57. A therapeutic system for inducing tolerance to an antigen in a patient, the system comprising a prostaglandin or agonist thereof, a PDE inhibitor, and the antigen or a derivative thereof.

58. A therapeutic system according to claim 57 wherein the prostaglandin or agonist thereof and/or the PDE inhibitor and/or the antigen or derivative thereof is in a preparation for administration locally at a site where tolerance is required.

59. A therapeutic system according to claim 57 wherein the prostaglandin or agonist thereof and/or PDE inhibitor and/or the antigen or derivative thereof is in a preparation for systemic administration.

60. A therapeutic system according to claim 59 wherein the prostaglandin or agonist thereof and/or PDE inhibitor and/or the antigen or derivative thereof is in a preparation for oral administration.

61. A therapeutic system according to claim 59 wherein the prostaglandin or agonist thereof and/or PDE inhibitor and/or the antigen or derivative thereof is formulated as a suppository or capsule.

62. A therapeutic system according to claim 61 wherein the suppository or capsule has an enteric coating for release of the prostaglandin or agonist thereof and/or PDE inhibitor and/or the antigen or derivative thereof in the bowel of the patient

63. A therapeutic system according to claim 57 wherein the prostaglandin or agonist thereof is any one of a prostaglandin E, prostaglandin E₂ or an analogue thereof, dinoprostone, gemeprost, misoprostol, alprostadil, limaprost, butaprost, 11-deoxy PGE1, AH23848, AH13205, 19-hydroxy PGE1 or 19-hydroxy PGE2.

64. A therapeutic system according to claim 57 wherein the prostaglandin is a 19-hydroxy PGE.

65. A therapeutic system according to claims 57 wherein the PDE inhibitor is selective for any of type IV, VII or VIII PDE.

66. A therapeutic system according to claim 57 wherein the PDE inhibitor is any one of IBMX, pentoxifylline, rolipram, CP80 633, CP102 995, CP76 593, Ro-20-1724, theophylline or denbufylline.

67. A therapeutic system according to claim 57 wherein the PDE inhibitor is selective for type IV PDE.

68. A therapeutic system according to claim 67 wherein the PDE inhibitor selective for type IV PDE is any one of rolipram, CP80 633, CP102 995, CP76 593, Ro-20-1724, denbufylline, CDP840, RP73401, or RS33793.

69. A therapeutic system according to claim 57 for combating a disease or condition associated with transplant rejection.

70. A therapeutic system according to claim 69 wherein the disease or condition associated with transplant rejection comprises graft versus host disease or host versus graft disease.

71. A therapeutic system according to claim 69 wherein the prostaglandin or agonist thereof and/or PDE inhibitor and/or antigen or derivative thereof are for administration prior to the transplant.

72. A therapeutic system according to claim 69 wherein the antigen is HLA-A2.

73. A therapeutic system according to claim 57 for treating an autoimmune disease or condition.

74. A therapeutic system according to claim 73 wherein the antigen is a self-antigen.

75. A therapeutic system according to claim 73 wherein the autoimmune disease is pernicious anaemia, and the antigen is vitamin B₁₂; the disease is Addison's disease, and the antigen is adrenal antigen; the disease is IDDM, and the antigen is GAD, insulin, or IA-2; the disease is Goodpasture's syndrome or renal vasculitis, and the antigen is renal antigen or endothelial antigen; the disease is myasthenia gravis, and the antigen is the acetyl choline receptor; the disease is sympathetic ophthalmia, and the antigen is ocular antigen; the disease is MS, and the antigen is MBP, PLP, or MOG; the disease is autoimmune haemolytic anaemia, and the antigen is red cell antigen; the disease is idiopathic leucopenia, and the antigen is leukocyte antigen; the disease is ulcerative colitis, and the antigen is a food antigen or a viral antigen; the disease is dermatomyositis, and the antigen is smooth muscle antigen; the disease is scleroderma, and the antigen is a connective tissue antigen; the disease is mixed connective tissue disease, and the antigen is a connective tissue antigen; the disease is irritable bowel syndrome, and the antigen is a food antigen; the disease is SLE, and the antigen is a histone protein or immunoglobulin heavy chain; the disease is Hashimoto's disease, primary myxoedema or thyrotoxicosis, and the antigen is thyroid antigen; the disease is rheumatoid arthritis, and the antigen is type II collagen or an HSP; the disease is thyroiditis, and the antigen is thyroglobulin; the disease is Behcet's disease, and the antigen is Sag, HLA-B44, B51, or HSP65; the disease is Coeliac disease/Dermatitis herpetiformis, and the antigen is gliadin or the α fraction thereof; or the disease is demyelinating disease, and the antigen is myelin.

76. A therapeutic system according to claim 57 for treating an allergic disease or condition in the patient.

77. A therapeutic system according to claim 76 wherein the allergic disease or condition is allergic asthma.

78. A therapeutic system according to claim 76 wherein the antigen is a mite allergen, a dust allergen, a cat allergen, a dog allergen or a horse allergen.

79. A therapeutic system according to claim 57, wherein the tolerance to the antigen is to treat an aberrant or undesired immune or inflammatory response to the antigen in the patient.

80. A therapeutic system according to claim 79 wherein the aberrant or undesired immune or inflammatory response involves a deficiency in IL-10 production and/or an increase in IL-12 production.

81. A composition for inducing tolerance to an antigen in a patient comprising a prostaglandin or agonist thereof, a PDE inhibitor, and the antigen or a derivative thereof.

82. A composition according to claim 81 wherein the PDE inhibitor is a type IV selective PDE inhibitor.

83. A composition according to claim 81 wherein the prostaglandin is a 19 hydroxy PGE.

84. A composition according to claim 81 for use in medicine.

85. A pharmaceutical composition comprising the composition of claim 81 and a pharmaceutically acceptable carrier.

86. (canceled)

87. The use according to claim 27, wherein the prostaglandin or agonist thereof is present in the medicament.

88. The use according to claim 27, wherein the PDE inhibitor is present in the medicament.

89. The use according to claim 27, wherein the antigen or the derivative thereof is present in the medicament.

90. The use according to claim 27, wherein a combination of the prostaglandin or agonist thereof and the PDE inhibitor is present in the medicament.

91. The use according to claim 27, wherein a combination of the prostaglandin or agonist thereof and the antigen is present in the medicament.

92. The use according to claim 27, wherein a combination of the PDE inhibitor and the antigen or derivative thereof is present in the medicament.

93. The use according to claim 27, wherein a combination of the prostaglandin or agonist thereof, the PDE inhibitor, and the antigen or derivative thereof is present in the medicament.
